(19) United States
(12) Patent Application Publication
(10) Pub. No.: US 2025/0302279 A1
(43) Pub. Date: Oct. 2, 2025

(54) MEDICAL DEVICES AND SYSTEMS WITH A ROTATABLE SHAFT

(71) Applicant: Boston Scientific Scimed, Inc., Maple Grove, MN (US)

(72) Inventors: Lewis HOLMBERG, Woodbury, MN (US); Aaron ABBOTT, Columbia Heights, MN (US); Ross MANNI, Star Prairie, WI (US); Shannon BUSSING, Naples, FL (US); Kiara HERRO, Minneapolis, MN (US); Anna ARNTSEN, Duluth, MN (US); Greyson HAMMER, Milton, WI (US); Jenna KRAUSE, Hawthorn Woods, IL (US); Raymond ZHANG, Rochester, MN (US); Elena DeGENNARO, Darien, CT (US); David GIORGIS, Alexandria, VA (US); Alexandra SKIBICKI, Lockport, IL (US)

(73) Assignee: Boston Scientific Scimed, Inc., Maple Grove, MN (US)

(21) Appl. No.: 19/089,711

(22) Filed: Mar. 25, 2025

Related U.S. Application Data
(60) Provisional application No. 63/569,883, filed on Mar. 26, 2024.

(51) Int. Cl. A61B 1/00; A61B 90/00

(52) U.S. Cl. CPC A61B 1/00066; A61B 90/03; A61B 2090/035

(57) ABSTRACT

Medical devices and systems with a rotatable shaft are described herein. The disclosure includes a medical device. The medical device includes a shaft, a handle, and a rotation assembly. The rotation assembly is positioned at a distal end of the handle. A proximal portion of the shaft is fixedly coupled to the rotation assembly. The rotation assembly includes a guide member. The guide member is fixedly coupled to the handle. The rotation assembly also includes a rotational member. The rotational member is rotatable relative to the guide member and the handle. The shaft is configured to rotate with the rotational member.

### CROSS-REFERENCE TO RELATED APPLICATIONS

This application claims the benefit of priority to U.S. Provisional Application No. 63/569,883, filed on Mar. 26, 2024, which is incorporated by reference herein in its entirety.

### TECHNICAL FIELD

Aspects of the present disclosure generally relate to medical devices, system, and methods related thereto. In particular, some aspects relate to medical systems, devices, and methods for delivering an elongate device.

### BACKGROUND

Ureteroscopes typically feature a handle, a light source, an imaging device (e.g., a CMOS camera), a shaft, and a working channel extending through the shaft. A typical working channel diameter may be approximately 1 mm and the typical working channel distal opening may be approximately 1 mm in diameter. The diameters of the working channel and the distal opening limit the types of instruments and the number of instruments that may be passed through the working channel and the distal opening. The shaft of an ureteroscope may include articulation joints for deflecting the shaft within a bodily lumen in order to aid in navigation and positioning of the distal opening of the shaft. The articulation joints typically are deflectable along one spatial plane, which may lead to increased difficulties in handling the ureteroscope. Increased handling difficulties may result in safety concerns, procedural complications, and delays during a medical procedure, among other difficulties.

The devices, systems and related methods of this disclosure may rectify some of the deficiencies described above or address other aspects of the prior art.

### SUMMARY

Each of the aspects disclosed herein may include one or more of the features described in connection with any of the other disclosed aspects.

For example, this disclosure includes a medical device. The medical device may include a shaft, a handle, and a rotation assembly. The rotation assembly may be positioned at a distal end of the handle. A proximal portion of the shaft may be fixedly coupled to the rotation assembly. The rotation assembly may include a guide member. The guide member may be fixedly coupled to the handle. The rotation assembly may include a rotational member. The rotational member may be rotatable relative to the guide member and the handle. The shaft may be configured to rotate with the rotational member.

The medical device may include one or more of the following features. A proximal end of the rotational member may include a first protrusion extending radially outward relative to a central longitudinal axis of the rotational member. The guide member may include a chamber. The chamber may be configured to receive the first protrusion of the rotational member. The guide member may include a first guide component and a second guide component. The chamber may be formed from a first chamber portion of the first guide component and a second chamber portion of the second guide component. The second guide component may include a second protrusion. The second protrusion may extend radially inward within the chamber toward the central longitudinal axis of the rotational member. Rotation of the rotational member may rotate the first protrusion within the chamber. The second protrusion may be configured to limit rotation of the first protrusion within the chamber. The first guide component and the second guide component may be coupled to one another. The guide member may be fixedly coupled to a distal portion of the handle. The chamber may have a cylindrical shape.

The medical device may include one or more of the following features. A lumen of the rotational member may extend from the proximal end of the rotational member through the distal end of the rotational member along a central longitudinal axis of the rotational member. The proximal end of the rotational member may be positioned within the handle. The distal end of the rotational member may extend distally beyond a distalmost end of the handle. The rotational member may include an O-ring and a recess. The recess may extend along a circumference of the rotational member. The recess may be configured to receive the O-ring therein. The chamber may include a second protrusion. The second protrusion may extend radially inward relative to a central longitudinal axis of the rotational member. The second protrusion may be positioned within the chamber to limit rotation of the first protrusion within the chamber so as to limit the rotation of the rotational member relative to the guide member to less than 360 degrees of rotation. The lumen of the rotational member may be configured to receive a medical instrument.

In another example, the disclosure includes a medical device. The medical device may include a first shaft and a handle. A proximal end of the first shaft may extend from a distal end of the handle. The medical device may include a rotation assembly. The rotation assembly may include a guide member. The guide member may include a chamber and a rotational member. The rotational member may be rotatable relative to the guide member. The rotational member may include a grippable portion, a second shaft, and a recess. The recess may be positioned between the grippable portion and the second shaft. The rotational member may include a lumen extending from the distal end of the rotational member to a proximal end of the rotational member. The second shaft may include a first protrusion extending (i) radially outward relative to the central longitudinal axis of the rotational member and (ii) within the chamber. A portion of the first shaft may be received within the lumen of the rotational member. The first shaft may be configured to rotate within the rotational member relative to the guide member and the handle.

The medical device may include one or more of the following features. The rotation assembly may include an O-ring. The recess may extend along a circumference of the rotational member. The recess may be sized and shaped to receive the O-ring. The grippable portion may extend distally beyond a distalmost end of the handle. The recess and the second shaft may be positioned within the handle proximal to the distalmost end of the handle. The guide member may include a second protrusion extending radially inward, relative to the central longitudinal axis of the rotational member, within the chamber. The second protrusion may be configured to limit rotation of the first protrusion within the chamber.

In yet another example, the disclosure includes a medical device. The medical device may include a shaft, a handle, and rotation assembly positioned at a distal portion of the handle. A proximal portion of the shaft may be fixedly coupled to the rotation assembly. The rotation assembly may include a guide member fixedly coupled to the handle and a rotational member. The rotational member may be rotatable relative to the guide member and the handle. The shaft may be fixedly coupled to the rotational member and may be configured to rotate with the rotational member. The guide member may include a chamber and a first protrusion extending radially inward within the chamber. The rotational member may include a second protrusion. The second protrusion may be positioned within the chamber, may extend radially outward from the rotational member, and may be configured to rotate within the chamber. The first protrusion may be configured to limit the rotation of the second protrusion within the chamber.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts a side view of an exemplary insertion device.

FIG. 2 depicts a perspective view of a portion of the insertion device of FIG. 1, with a portion of the insertion device removed to expose interior components of the device.

FIG. 3A depicts perspective view of a rotation assembly of the insertion device, the rotation assembly including a first guide component, a second guide component, and a rotational member.

FIG. 3B depicts an exploded view of the rotational assembly of FIG. 3A.

FIG. 4 depicts a perspective view of the rotational member of FIGS. 3A and 3B.

FIGS. 5A and 5B depict perspective views of the first guide component of FIGS. 3A and 3B.

FIGS. 6A and 6B depict perspective views of the second guide component of FIGS. 3A and 3B.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. In some of the drawings, arrows labeled “P” and “D” label proximal and distal directions, respectively.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although ureteroscopes are referenced herein for illustration purposes, it will be appreciated that the disclosure encompasses any suitable medical device configured to allow an operator to access and/or view internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

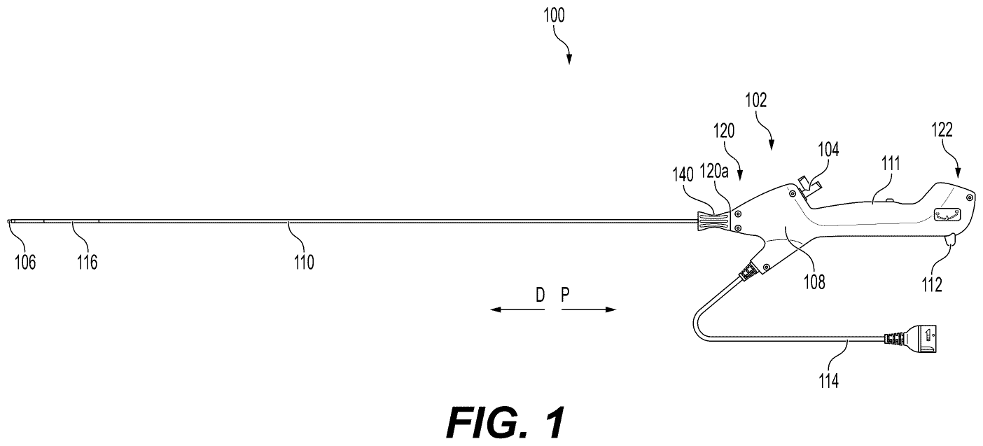

FIG. 1 illustrates a system 100 including an insertion device 102. The insertion device 102 may be an ureteroscope (e.g., LithoVue™ Single-Use Digital Flexible Ureteroscope by Boston Scientific Corp.), an endoscope, a hysteroscope, a bronchoscope, a cystoscope, or any other similar device. The insertion device 102 may include a handle 108, a delivery shaft 110, and an actuator 112 on handle 108. In some examples, actuator 112 may be for steering a distal end 106 of the delivery shaft 110. Actuator 112 may be a lever, knob, button, or any other suitable actuator. A port 104, which could be a T-connector as shown, may internally connect one or more lumens (not shown) extending through the delivery shaft 110 to one or more distal openings in distal end 106 such that various medical devices or other material (e.g. a fluid) may be inserted through the delivery shaft 110 of the insertion device 102. The insertion device 102 may include an integral camera at distal end 106 that is connected to processing software and/or a display via a communication and power conduit (e.g. umbilicus 114 or wirelessly). In other examples, power may be received separately from a communication conduit.

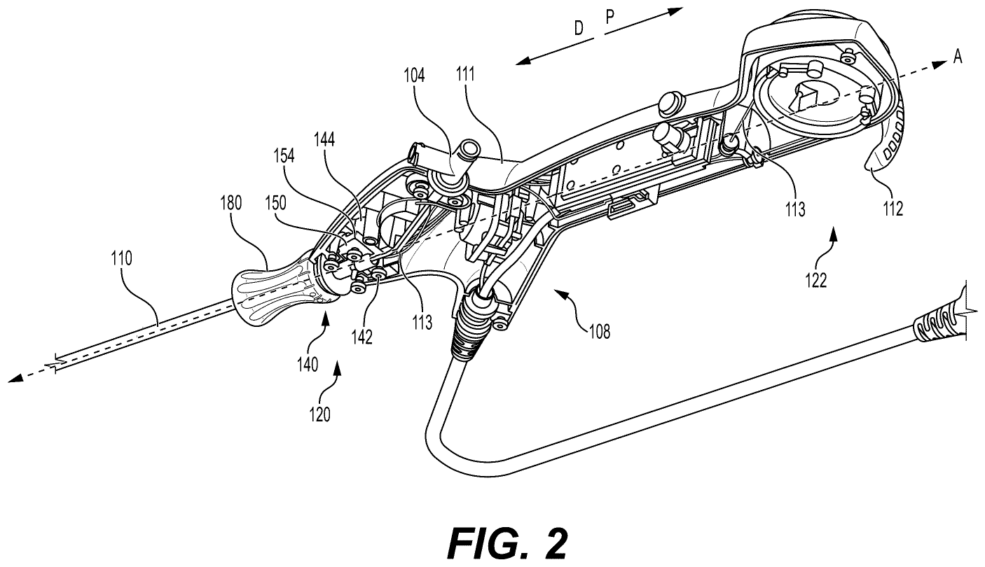

As seen in FIG. 2, the handle 108 may include a distal end 120 (e.g., a distal portion) and a proximal end 122 (e.g., a proximal portion). It should be understood that certain components (e.g., a portion of a shell 111 of handle 108, a portion of guide member 150, accessory tubes, medical instruments, etc.) of insertion device 102 are omitted from FIG. 2 for clarity purposes. For example, a portion of handle 108 is removed in FIG. 2 to expose interior components of handle 108. Delivery shaft 110 may extend distally from distal end 120. A proximal end (not shown) of delivery shaft 110 may be positioned within distal end 120. In some examples, a proximal end of delivery shaft 110 may be positioned within rotation assembly 140, and/or may be positioned within handle 108.

Insertion device 102 may include a rotation assembly 140. Delivery shaft 110 (such as the proximal end of shaft 110) may be fixedly coupled to/affixed to rotation assembly 140 and rotatable with a portion/component of rotation assembly 140. Rotation assembly 140 may facilitate clockwise and counter-clockwise rotation of shaft 110 about a central longitudinal axis of insertion device 102 (e.g., axis A as shown in FIG. 2) and/or a central longitudinal axis of shaft 110, and rotation of shaft 110 via rotation assembly may be relative to handle 108. Rotation assembly 140 may provide shaft 110 with approximately 360 degrees of rotation (e.g., approximately 360 degrees of angular displacement) about axis A.

Referring to FIG. 1, shaft 110 may include one or more articulation joints 116 (or an articulation portion) to facilitate deflection of shaft 110. One or more articulation joints 116 may be actuated/steered by actuator 112, for example via a pull wire 113 operably connecting actuator 112 to one or more articulation joints 116. In other examples, articulation joints 116 may be actuated via a remote system connected, wirelessly or via one or more wires, to insertion device 102. The one or more articulation joints 116 may provide shaft 110 with approximately 270 degrees of bending capability. Articulation joints 116 may deflect shaft 110 up and down relative to a longitudinal axis of a proximal portion of shaft 110. Articulation joints 116 and rotation assembly 140 may be utilized together by a user to provide shaft 110 with a full 360 degrees of rotation during operation. For example, shaft 110 may be deflected upward and may be rotated relative to handle 108 via rotation assembly 140, providing approximately 360 degrees of rotation. Similarly, shaft 110 may be deflected downward and may be rotated via rotation assembly 140, providing approximately 360 degrees of rotation. The deflection of articulation joints 116 and the rotation of rotation assembly 140 provide a combined full 360 degrees of rotation to distal end 106. For example, a distal front face (not shown) of distal end 106 may face the direction of central longitudinal axis A when insertion device 102 is in a neutral position (e.g., when articulation joints 116 are in a straight position aligned with central longitudinal axis A, shown in FIG. 1). By either bending one or more articulation joints 116 and/or rotating rotation assembly 140, a user may rotate the distal front face of distal end 106 to face any direction (e.g., provide 360 degrees of rotation). Rotation assembly 140 will be described in further detail below.

As shown in FIG. 2, rotation assembly 140 may be positioned within handle 108. For example, rotation assembly 140 may be positioned within handle 108 at distal end 120 and/or at least a portion of rotation assembly 140 may be proximal to a distalmost end 120a of handle 108. Rotation assembly 140 may be coupled to handle 108 via one or more fasteners 142, each with corresponding hole(s) (e.g., through holes or blind holes) defined by handle 108. In other examples, rotation assembly 140 may not include fasteners 142 and may be coupled to handle 108 via another means, such as via glue, press-fit between one or more supports of the handle, or any other coupling means known in the art. Handle 108 may include one or more mount bosses 144 configured to help anchor rotation assembly within handle 108. Mount boss(es) 144 may each define one or more holes (not shown) for receiving one of fastener(s) 142. Fasteners 142 may be threaded/inserted through one or more holes (e.g., holes 210, 212) of guide components 152, 154 and the hole(s) of mount boss(es) 144 to fix guide components 152, 154 within handle 108.

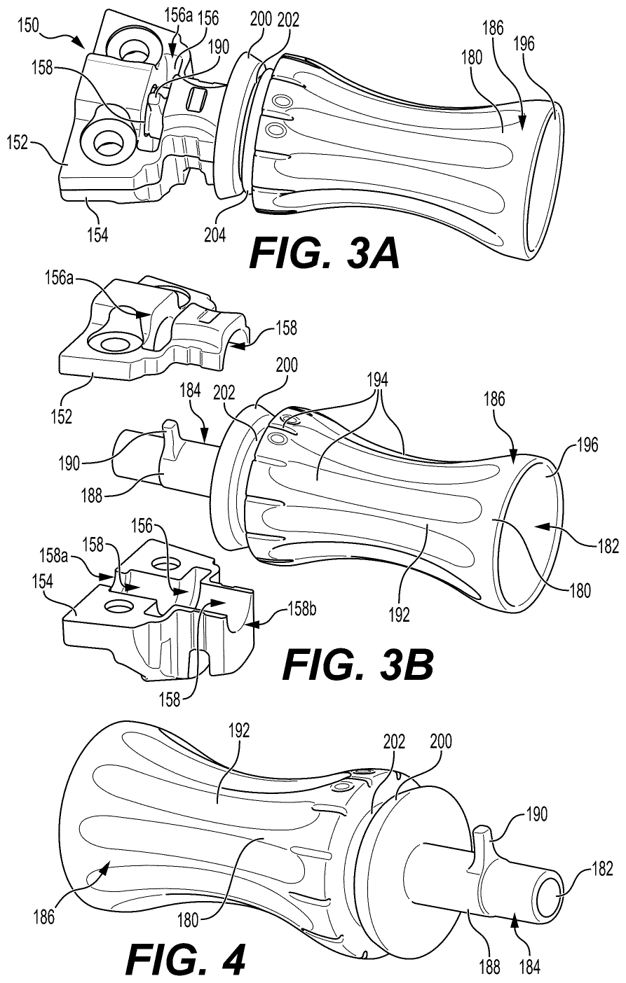

FIGS. 3A-3B depict rotation assembly 140, and FIGS. 4-6B show various portions of rotation assembly 140. Rotation assembly 140 may include a guide member 150 and a rotational member 180. Rotational member 180 may rotate relative to guide member 150. Rotational member 180 may be fixedly or releasably coupled to a proximal end of shaft 110, and rotational member 180 may be rotatably coupled to guide member 150.

As shown in FIGS. 3A-3B, guide member 150 may be formed from one or more guide components such a first guide component 152 and second guide component 154. First guide component 152 and second guide component 154 may be sized and shaped to fit within distal end 120 of handle 108 (for example, a distal end of a handle of LithoVue™ Single-Use Digital Flexible Ureteroscope by Boston Scientific Corp.). Guide member 150 may include a chamber 156. Chamber 156 may include an opening 156a. Chamber 156 may be sized and shaped to receive at least a portion of rotational member 180. Chamber 156 may have a cylindrical shape. Guide member 150 may include a lumen 158, and chamber 156 may be formed by portions of guide member 150 forming lumen 158. Lumen 158 may extend along axis A. Lumen 158 may include an opening 158a at a proximal-most end of guide member 150 and an opening 158b at a distalmost end of guide member 150. A diameter of opening 158b may be less than a diameter of a portion of lumen 158 distal to opening 158b. Lumen 158 may be in communication with chamber 156. Lumen 158 may extend proximally from chamber 156 and may extend distally from chamber 156. A diameter of lumen 158 distal to chamber 156 may be larger than a diameter of lumen 158 proximal to chamber 156. It should be understood that first guide component 152 and second guide component 154 may define portions of chamber 156 and lumen 158.

As shown in FIGS. 3A, 3B, and 4, rotational member 180 may include a lumen 182 extending along a central longitudinal axis of rotational member 180 and/or axis A from a distal end of rotational member 180 to a proximal end of rotational member 180. At least a portion of shaft 110 may be received within lumen 182. One or more lumens (not shown) of shaft 110 may be connected to rotational member 180 at a distal end of lumen 182 and may be in fluid communication with lumen 182. Lumen 182 may be sized and shaped to receive a portion of a medical instrument, pull wire 113, and one or more electrical conduits extending from within handle 108 to distal end 106 of shaft 110. A portion of a medical instrument may be extended from port 104 through lumen 182 to the lumen of shaft 110. Additionally, pull wire 113 and electrical conduits may be extended from within handle 108 through lumen 182 and one or more lumens of shaft 110. Rotational member 180 may include a first, proximal portion 184 and a second, distal portion 186.

Proximal portion 184 may include a shaft 188. Shaft 188 may extend along axis A and may include a portion of lumen 182. Proximal portion 184 may include a protrusion 190. Protrusion 190 may extend radially outward, relative to axis A, from an exterior surface of shaft 188. Protrusion 190 may decrease in a diameter or other dimension as protrusion 190 extends radially outward, and may be tapered. A diameter of a portion of shaft 188 proximal to protrusion 190 may be less than a diameter of a portion of shaft 188 distal to protrusion 190. The diameter of the portion of shaft 188 proximal to protrusion 190 may be larger than a diameter of opening 158a to prevent rotational member 180 from sliding in a proximal direction within lumen 158. Relatedly, a diameter of a portion of lumen 158 proximal to chamber 156 may be less than a diameter of a portion of lumen 158 distal to chamber 156.

Distal portion 186 may include a grippable portion 192. Grippable portion 192 may be configured to receive one or more digits (e.g., fingers) of an operator. Grippable portion 192 may extend distally beyond distalmost end 120a of handle 108, and may be entirely distal from handle 108 when rotation assembly 140 is coupled to handle 108. In the proximal to distal direction, grippable portion 192 may decrease in diameter to a minimum diameter and then may increase in diameter so as to resemble an hourglass shape, and may include one or more convex surfaces at a radially-outermost portion of grippable portion 192. Grippable portion 192 may include one or more grooves 194 extending longitudinally along grippable portion 192 relative to axis A. One or more grooves 194 may help one or more digits of the operator to rotate rotational member 180. Distal portion 186 may include a cavity 196. Cavity 196 may include a semi-spherical shape. Cavity 196 may be positioned at a distalmost end of distal portion 186. Cavity 196 may be in communication with lumen 182. Cavity 196 may be configured to help an operator in guiding and inserting shaft 110 into lumen 182 (e.g., a distal opening of lumen 182). Cavity 196 may provide strain relief for shaft 110 and may help prevent kinking of shaft 110.

Rotational member 180 may include middle portion 200 positioned between distal portion 186 and proximal portion 184. Middle portion 200 may include a recess 202. Recess 202 may extend along a circumference of middle portion 200. Recess 202 may be sized and shaped to receive an O-ring 204 within. O-ring 204 may be formed from rubber or similar materials. O-ring 204 may provide friction between O-ring 204 and a portion of handle 108 or other component of system 100 so as to prevent unwanted rotation of rotational member 180 during a procedure. O-ring 204 may contact a portion of handle 108 to prevent unwanted rotation. In some examples, rotational member 180 may not include O-ring 204.

FIGS. 3A and 3B show an assembled view of rotation assembly 140 (FIG. 3A) and an exploded view of rotation assembly 140 (FIG. 3B). When assembled, proximal portion 184 may be positioned within chamber 156 and lumen 158. Protrusion 190 may be positioned within chamber 156 and configured to rotate within chamber 156. Shaft 188 may be positioned within lumen 158 and configured to rotate within lumen 158. It should be understood that rotation of grippable portion 192 (clockwise or counter-clockwise) may rotate protrusion 190 within chamber 156 (clockwise or counter-clockwise).

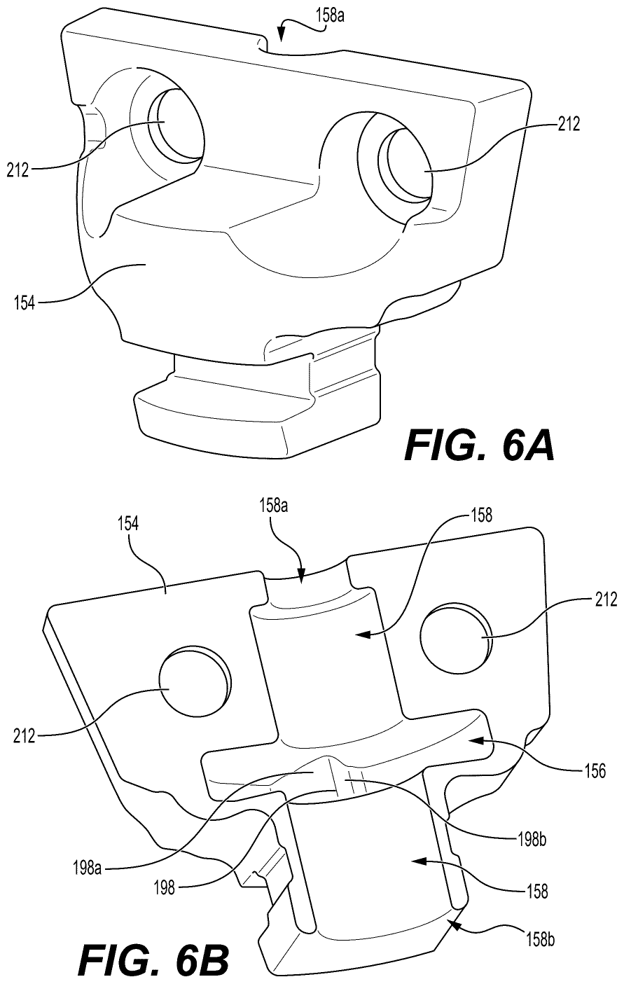

Chamber 156 may include a protrusion 198 (as shown in FIG. 6B). Protrusion 198 may extend radially inward relative to axis A from an inner surface of chamber 156. Protrusion 198 may extend from second guide component 154, however it should be understood that protrusion 198 may extend from first guide component 152 or second guide component 154. Protrusion 198 may decrease in a dimension/diameter as protrusion 198 extends radially inward relative to axis A toward a center of chamber 156. Protrusion 198 may include a first surface 198a and a second surface 198b, and first surface 198a may be angled relative to second surface 198b. Protrusion 198 may be configured to block or limit the rotation of protrusion 190 within chamber 156 so as to prevent protrusion 198 from completing a full revolution relative to axis A. Preventing a full-revolution may be desirable to prevent damage to the medical instruments, electrical conduits, and/or pull wires (such as pull wire 113) extending through lumen 182 and the one or more lumens of shaft 110. The angle between first surface 198a and second surface 198b reduces the maximum angular displacement of protrusion 190 within chamber 156 to less than 360 degrees of angular displacement. For example, the angle between first surface 198a and second surface 198b may be any suitable angle, and may be configured to limit angular displacement of protrusion 190 within chamber 156 to any degree of angular displacement less than 360 degrees, such as 355 degrees, 350 degrees, 345 degrees, or any other suitable maximum angular displacement less than 360 degrees.

Rotational member 180 of rotation assembly 140 may include a neutral configuration. In the neutral configuration, protrusion 190 may be positioned opposite side of chamber 156 from protrusion 198 and/or a longitudinal axis of protrusion 190 may be parallel with a longitudinal axis of protrusion 198. From the neutral configuration, rotational member 180 may be rotated clockwise or counter-clockwise until protrusion 190 contacts protrusion 198. From the neutral configuration, protrusion 190 may be rotated one direction (e.g., clockwise or counter-clockwise) approximately 180 degrees, and angular displacement may be limited by the angle between first surface 198a and second surface 198b.

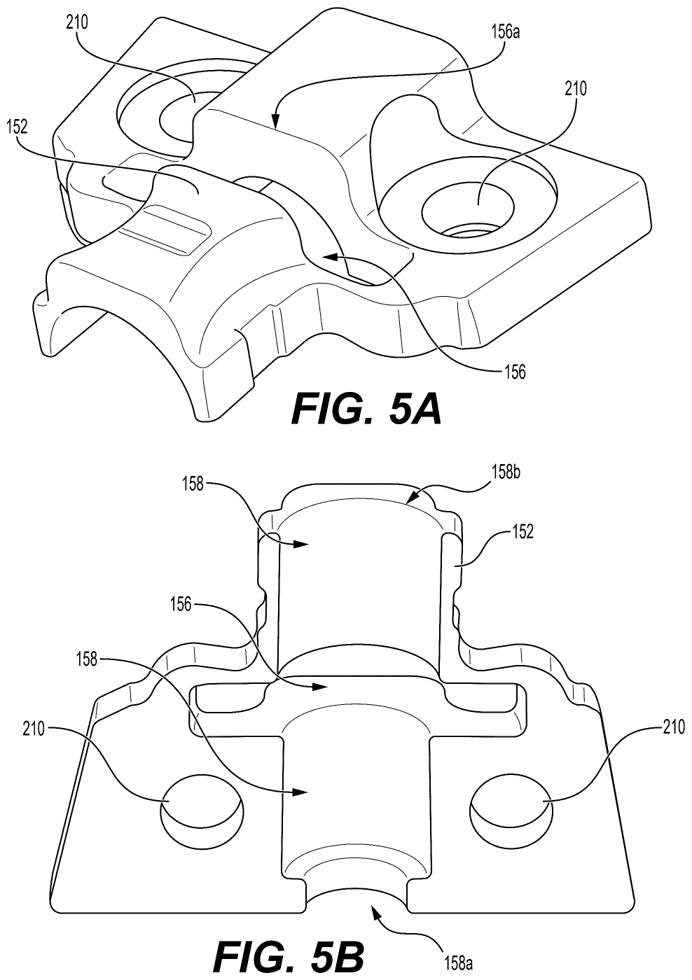

FIGS. 5A-5B show first guide component 152. First guide component 152 may define a portion of chamber 156 (e.g., a first chamber portion). Chamber 156 includes opening 156a. Opening 156a may provide clearance for protrusion 190 to rotate freely within chamber 156 but for protrusion 198. First guide component 152 may include one or more holes 210. One or more holes 210 may be sized and shaped to receive a fastener therein (for example, fastener(s) 142). One or more holes 210 may be through holes. In some examples, first guide component 152 may not include holes 210.

FIGS. 6A-6B show second guide component 154. Second guide component 154 may define a portion of chamber 156 (e.g., a second chamber portion). Second guide component 154 may include one or more holes 212. In some examples, second guide component 154 may not include holes 212. First and second guide components 152, 154 may include an equal number of holes 210, 212, respectively. Accordingly, each of one or more holes 210 may correspond to one of one or more holes 212. One or more holes 212 may be sized and shaped to receive a fastener therein (for example, fasteners 142). One or more holes 212 may be through holes. In some examples, second guide component 154 may not include holes 212.

First guide component 152 and second guide component 154 may be fastened to one another by inserting fastener(s) (e.g., fastener(s) 142) through each of one or more holes 210 and then through a corresponding hole of one or more holes 212. After inserting fastener(s) 142, fastener(s) 142 may be tightened or otherwise fastened to affix first guide component 152 to second guide component 154. After inserting fastener(s) 142 through holes 210, 212, fastener(s) 142 may be tightened to fasten first guide component 152 and second guide component 154 to one another. In some examples, after being inserted through holes 210, 212, fastener(s) 142 may be further inserted into the one or more corresponding holes of mount bosses 144, and tightening fastener(s) 142 may fix the first guide component 152 and second guide component 154 to handle 108 (in other words, first guide component 152 and second guide component 154 may be fixedly coupled to distal end 120 of handle 108). In other examples, first guide component 152 may be glued or otherwise fastened to second guide component 154 without fastener(s), and/or guide member 150 may be glued or otherwise fastened to handle 108 without fastener(s).

When insertion device 102 is used in an exemplary medical procedure, the operator may take the initial step of extending a medical instrument through port 104, lumen 182, and one or more lumens of delivery shaft 110 to distal end 106 of delivery shaft 110. The operator may also take the initial step of positioning/navigating distal end 106 of delivery shaft 110 within a bodily lumen or artificial orifice near a treatment site. While positioning/navigating the distal end 106 of delivery shaft 110, it may be desirable to deflect and/or rotate delivery shaft 110. For example, the operator may rotate rotational member 180 (and by extension delivery shaft 110) clockwise or counter-clockwise to a desired angle by manipulating grippable portion 192. In some examples, shaft 110 may remain at the desired angle after the operator releases rotational member 180, due to the friction between O-ring 204 and handle 108. The operator may deflect the delivery shaft 110 to a desired angle/deflection via actuator 112 which is operably coupled articulation joints 116. It should be understood, that the operator may rotate rotational member 180 or deflect the delivery shaft 110 as desired during the procedure in order to reposition the distal end 106 of the delivery shaft 110.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a shaft;
a handle; and
a rotation assembly positioned at a distal end of the handle, wherein a proximal portion of the shaft is fixedly coupled to the rotation assembly, the rotation assembly comprising:
a guide member fixedly coupled to the handle, and
a rotational member, wherein the rotational member is rotatable relative to the guide member and the handle;
wherein the shaft is configured to rotate with the rotational member.

2. The medical device of claim 1, wherein a proximal end of the rotational member includes a first protrusion extending radially outward relative to a central longitudinal axis of the rotational member.

3. The medical device of claim 2, wherein the guide member includes a chamber, wherein the chamber is configured to receive the first protrusion of the rotational member.

4. The medical device of claim 3, wherein the guide member includes a first guide component and a second guide component, and wherein the chamber is formed from a first chamber portion of the first guide component and a second chamber portion of the second guide component.

5. The medical device of claim 4, wherein the second guide component includes a second protrusion; wherein the second protrusion extends radially inward within the chamber toward the central longitudinal axis of the rotational member.

6. The medical device of claim 5, wherein rotation of the rotational member rotates the first protrusion within the chamber.

7. The medical device of claim 6, wherein the second protrusion is configured to limit rotation of the first protrusion within the chamber.

8. The medical device of claim 7, wherein the first guide component and the second guide component are coupled to one another, wherein the guide member is fixedly coupled to a distal portion of the handle.

9. The medical device of claim 7, wherein the chamber has a cylindrical shape.

10. The medical device of claim 3, wherein a lumen of the rotational member extends from the proximal end of the rotational member through the distal end of the rotational member along a central longitudinal axis of the rotational member.

11. The medical device of claim 10, wherein the proximal end of the rotational member is positioned within the handle and the distal end of the rotational member extends distally beyond a distalmost end of the handle.

12. The medical device of claim 11, wherein the rotational member includes an O-ring and a recess, wherein the recess extends along a circumference of the rotational member, wherein the recess is configured to receive the O-ring therein.

13. The medical device of claim 12, wherein the chamber includes a second protrusion, wherein the second protrusion extends radially inward relative to the central longitudinal axis of the rotational member.

14. The medical device of claim 13, wherein the second protrusion is positioned within the chamber to limit rotation of the first protrusion within the chamber so as to limit the rotation of the rotational member relative to the guide member to less than 360 degrees of rotation.

15. The medical device of claim 14, wherein the lumen of the rotational member is configured to receive a medical instrument.

16. A medical device comprising:
a first shaft;
a handle; wherein a proximal end of the first shaft extends from a distal end of the handle; and
a rotation assembly, comprising:
a guide member including a chamber, and
a rotational member, wherein the rotational member is rotatable relative to the guide member, wherein the rotational member includes a grippable portion, a second shaft, and a recess, wherein the recess is positioned between the grippable portion and the second shaft, wherein the rotational member includes a lumen extending from a distal end of the rotational member to a proximal end of the rotational member;
wherein the second shaft includes a first protrusion extending (i) radially outward, relative to a central longitudinal axis of the rotational member, and (ii) within the chamber;
wherein a portion of the first shaft is received within the lumen of the rotational member; and
wherein the first shaft is configured to rotate with the rotational member relative to the guide member and the handle.

17. The medical device of claim 16, wherein the rotation assembly further comprises an O-ring, wherein the recess extends along a circumference of the rotational member, wherein the recess is sized and shaped to receive the O-ring.

18. The medical device of claim 17, wherein the grippable portion extends distally beyond a distalmost end of the handle, wherein the recess and the second shaft are positioned within the handle proximal to the distalmost end of the handle.

19. The medical device of claim 18, wherein the guide member includes a second protrusion extending radially inward, relative to the central longitudinal axis of the rotational member, within the chamber; wherein the second protrusion is configured to limit rotation of the first protrusion within the chamber.

20. A medical device comprising:
a shaft;
a handle; and
a rotation assembly positioned at a distal portion of the handle, wherein a proximal portion of the shaft is fixedly coupled to the rotation assembly, the rotation assembly comprising:
a guide member fixedly coupled to the handle, and
a rotational member, wherein the rotational member is rotatable relative to the guide member and the handle;
wherein the shaft fixedly coupled to the rotational member and is configured to rotate with the rotational member;
wherein the guide member includes a chamber and a first protrusion extending radially inward within the chamber;
wherein the rotational member includes a second protrusion, wherein the second protrusion is positioned within the chamber, extends radially outward from the rotational member, and is configured to rotate within the chamber;
wherein the first protrusion is configured to limit the rotation of the second protrusion within the chamber.
